(19) United States
(12) Patent Application Publication
(10) Pub. No.: US 2008/0228084 A1
(43) Pub. Date: Sep. 18, 2008

(54) Method and Apparatus for Reduction of Spurious Effects on Physiological Measurements

(76) Inventors: Michel Bedard, Quebec (CA); Dany Nolet, Quebec (CA)

Correspondence Address: SEYFARTH SHAW LLP, 131 S. DEARBORN ST., SUITE 2400, CHICAGO, IL, 60603-5803, US

(21) Appl. No.: 11/666,938

(22) PCT Filed: Nov. 9, 2005
(86) PCT No.: PCT/CA2005/001710

§ 371 Date: Nov. 2, 2007

Related U.S. Application Data
(60) Provisional application No. 60/625,957, filed on Nov. 9, 2004.

(51) Int. Cl. A61B 5/00

(52) U.S. Cl. 600/477; 600/300

(57) ABSTRACT

A method and apparatus for reducing motion artifact and spurious noise effects when computing estimates of values representative of at least one physiological parameter of a subject. For motion, measured motion values are compared with a motion threshold and the taking of physiological measurements used for computing the physiological parameter estimate values are either suspended until a measured motion value is under the threshold or a correction function is applied to the physiological measurements, the correction function being based on the measured motion values. As for spurious noise, physiological measurements taken while emitters are turned off are subtracted from physiological measurements taken while emitters are turned on in order to eliminate outside noise contamination.

### TECHNICAL FIELD

The present invention relates to a method and apparatus for the reduction of spurious effects on physiological measurements. More specifically, the present invention relates to a method and apparatus for the reduction of motion artifact and spurious noise effects on physiological measurements.

### BACKGROUND

There is a great potential for applying optical technologies to biology, medicine and sports to track various physiological parameters or states and provide real time information to the user or to medical personnel. While many studies have shown this great potential, very few concrete products using optical technologies have been developed or marketed. Some of the reasons for this are the difficulty to isolate a signal of interest from the various interferences that come from the external environment, the fact that the measurements must be made in a continuous manner on a constantly moving subject and to the variable nature of the human body itself.

The elastic nature of human tissue complicates the taking of optical measurements when a subject is in motion since tissue compression and expansion instantly affect the optical properties of the tissue while the signal of interest remains fairly constant.

A complication that comes with the use of portable measurement devices is that the nature and the sources of the noises are constantly changing. Noise sources are present in both the measurement device itself and the external environment. Electrical noises from AC lines or surrounding electronic devices are obvious noise sources. Optical noise coming from the sun or from artificial lights may migrate into the skin and through the optical sensors. Both the electric and the optical noises may vary over time and with the motion of the subject.

In the present specification, there is described a method and apparatus designed to overcome the above-described limitations.

### SUMMARY

The present invention relates to a method for reducing motion artifact when computing estimates of values representative of at least one physiological parameter of a subject, comprising the steps of measuring a motion value and comparing the motion value with a motion threshold. If the compared motion value is lower than the motion threshold then taking at least one physiological measurement, estimating the values representative of the at least one physiological parameter by applying a mathematical model to the at least one physiological measurement and providing the estimate of the values representative of the at least one physiological parameter.

The present invention also relates to a method for reducing motion artifact when computing estimates of values representative of at least one physiological parameter of a subject, comprising the steps of repeatably measuring a motion value and comparing each motion value with a motion threshold. If the compared motion value is lower than the motion threshold then taking at least one physiological measurement, estimating the values representative of the at least one physiological parameter by applying a mathematical model to the at least one physiological measurement and providing the estimates of the values representative of the at least one physiological parameter. If not, after a predetermined number of consecutive compared motion values that are higher than the motion threshold then providing a warning to the subject.

The present invention further relates to a method for reducing motion artifact when computing estimates of values representative of at least one physiological parameter of a subject, comprising the steps of measuring a motion value, taking at least one physiological measurement, applying a correction function to the at least one physiological measurement, the correction function being based on the measured motion value, estimating the values representative of the at least one physiological parameter by applying a mathematical model to the at least one corrected physiological measurement and providing the estimates of the values representative of the at least one physiological parameter.

The present invention further still relates to a method for reducing spurious noise when computing estimates of values representative of at least one physiological parameter of a subject, comprising the steps of generating a probing signal comprising at least one wavelength, propagating the probing signal from a propagation point, measuring reflectance values of the probing signal for a subset of the at least one wavelength from at least two distances from the propagation point, shutting off the probing signal for the subset of the at least one wavelength, measuring a shut-off reflectance value from the at least two distances from the propagation point, computing adjusted reflectance values by subtracting the shut-off reflectance values from the reflectance values, estimating the values representative of the at least one physiological parameter by applying a mathematical model to adjusted reflectance values and providing the estimates of the values representative of the at least one physiological parameter.

The present invention also relates to an apparatus implementing the above described methods.

The foregoing and other objects, advantages and features of the present invention will become more apparent upon reading of the following non restrictive description of illustrative embodiments thereof, given by way of examples only with reference to the accompanying drawings.

### BRIEF DESCRIPTION OF THE FIGURES

Non-limitative illustrative embodiments of the invention will now be described by way of examples only with reference to the accompanying drawings, in which:

FIG. 1 which is labeled “Prior Art”, is a block diagram showing an apparatus for the monitoring of skin parameters;

FIG. 2 is a block diagram showing an apparatus for the monitoring of skin parameters similar to FIG. 1 but with a motion sensor;

FIG. 3 is a flow diagram of an algorithm for the monitoring of skin parameters;

FIG. 4 is a flow diagram of an algorithm for the monitoring of skin parameters with motion artifact reduction;

FIG. 5 is a flow diagram of an algorithm for setting a motion threshold;

FIG. 6 is a flow diagram of an alternative algorithm for the monitoring of skin parameters with motion artifact reduction;

FIG. 7 is a flow diagram of an algorithm for setting a motion correction factor;

FIG. 8 is a flow diagram of an algorithm for the monitoring of skin parameters with spurious noise reduction;

FIG. 9 is a flow diagram of an algorithm for the monitoring of skin parameters with motion artifact reduction and spurious noise reduction;

FIG. 10 shows integrating amplifier waveforms; and

FIG. 11 shows transimpedance amplifier waveforms.

### DETAILED DESCRIPTION

Generally stated, a method and apparatus according to an illustrative embodiment of the present invention provide means to reduce the adverse effects of environmental conditions such as motion artifact and spurious noise effects on physiological measurements used to compute estimates of physiological parameters, for example skin parameters.

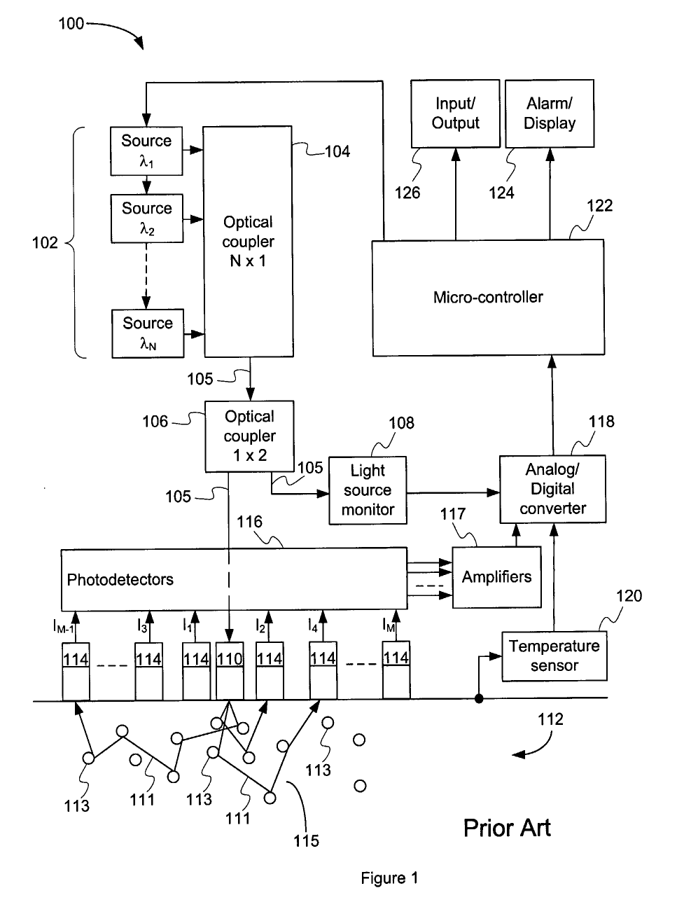

Referring to FIG. 1, an example of a monitoring apparatus 100 estimates skin parameters such as, for example, chromophore concentrations and scattering coefficient is illustrated. The monitoring apparatus 100 uses N light sources (or emitters) 102, each generating a light beam at respective predetermined wavelengths λ₁ to λ_{N}, coupled to a N×1 optical coupler 104 in order to generate a probing light beam 105 comprising all of the N wavelengths of the N individual light sources 102. The number of light sources 102, and thus wavelengths, as well as their power levels, may vary depending on the application.

The probing light beam 105 then goes through a 1×2 optical coupler 106 that provides the probing light beam 105 to both a light source monitor 108 and to an emitter collimating optic 110. The emitter collimating optic 110, advantageously positioned in direct contact with the skin, propagates the probing light beam 105 into the dermis 112 of the skin. The probing light beam 105 is then attenuated and scattered into a number of reflected beams 111 by various scatterers 113 and chromophores 115, which are present in the dermis. The attenuated and reflected beams 111 are then received by receiver collimating optics 114, providing optical signals I₁ to I_{M} to photodetectors 116. Each of the receiver collimating optics 114 is positioned at a distance away from the emitter collimating optic 110 that is different from that of the other receiver collimating optics 114. The number of receiver collimating optics 114 may vary according to the application. A temperature sensor 120 provides a signal indicative of the temperature of the skin.

An Analog to Digital Converter (ADC) 118 then converts the analog signals from the light source monitor 108, the photodetectors 116, as amplified by amplifiers 117, and the temperature sensor 120 into digital signals which are provided to a micro-controller 122. The micro-controller 122 includes an algorithm that controls the operations of the apparatus and performs the monitoring of certain clinical states, and may also perform estimations of certain biological or physiological parameters such as, for example, chromophore concentrations and scattering coefficient, which will be further described below. The results of the monitoring and estimations are then given to the wearer of the monitoring apparatus 100 by either setting a visual, audio and/or mechanical alarm, when a certain clinical state is detected, of displaying the result via alarm/display 124. The micro-controller 122 may also be connected to an input/output 126 through which data such as, for example, a reference blood glucose level may be provided to the monitoring apparatus 100 or through which data such as, for example, chromophore concentrations and scattering coefficient may be provided from the monitoring apparatus 100 to other devices. It is to be understood that the input/output 126 may be any type of interface such as, for example, an electrical, infrared (IR) or a radio frequency (RF) interface.

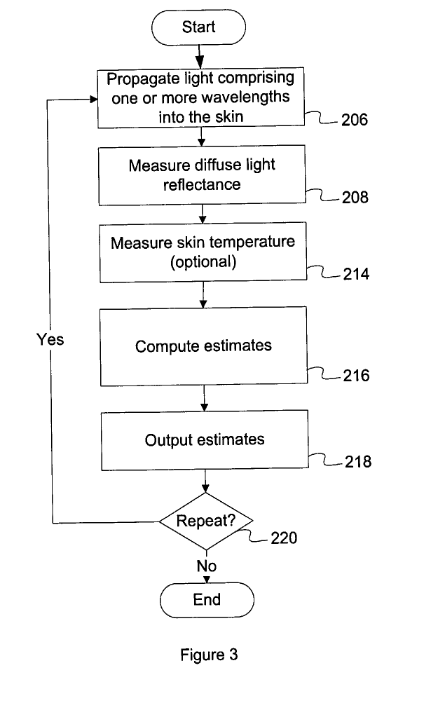

An example of an algorithm that may be executed by the micro-controller 122 is depicted by the flow chart shown in FIG. 3. The steps composing the algorithm are indicated by blocks 206 to 220.

At block 206 the algorithm starts by propagating light comprising one or more wavelengths into the skin, the wavelengths being selected according to the application of interest such that variations on light reflectance values at the input of the receiver collimating optics 114 may be observed as a function the variation of some estimated parameters.

At block 208, the diffuse light reflectance is measured at two or more distances from the source of the propagated light of block 206. The diffuse light reflectance measurements are advantageously taken simultaneously for all distances, the longer the time interval between each measurement, the less precise the algorithm results may become. The distances, as well as their values, are selected according to the application. The more distances are used, the more precise the diffuse light reflectance model becomes, but also the more computation intensive it becomes and more expensive becomes the associated estimation apparatus 100.

At block 214, which is optional, the skin temperature is measured.

Then, at block 216, the algorithm computes estimates of the desired physiological parameters using the reflectance measurements, and skin temperature if measured, and displays those estimates at block 218 using display/alarm 124. The algorithm may further detect clinical conditions using the estimated parameter values, in which case block 218 may also activate an alarm using display/alarm 124. It is to be noted that the parameter estimates and/or detection of clinical conditions may also be provided to another device for further processing using input/output 126. Following which, at block 220, the whole algorithm is repeated if continuous monitoring is desired, otherwise the algorithm ends.

Various environmental conditions may affect the photodetectors 116 readings of the reflected beams 111 received by receiver collimating optics 114, which readings are used at block 216 to compute estimates of the desired physiological parameters. One such condition is movement of the wearer of the device, which may cause motion artifacts between the apparatus and the skin and/or the skin and the underlying tissues. A second condition is spurious noise present in the reflected beam 111, such as caused by ambient lighting, to which possible electrical offsets from the photodetectors 116 or amplifiers 117 may be added.

### Motion Artifact Reduction

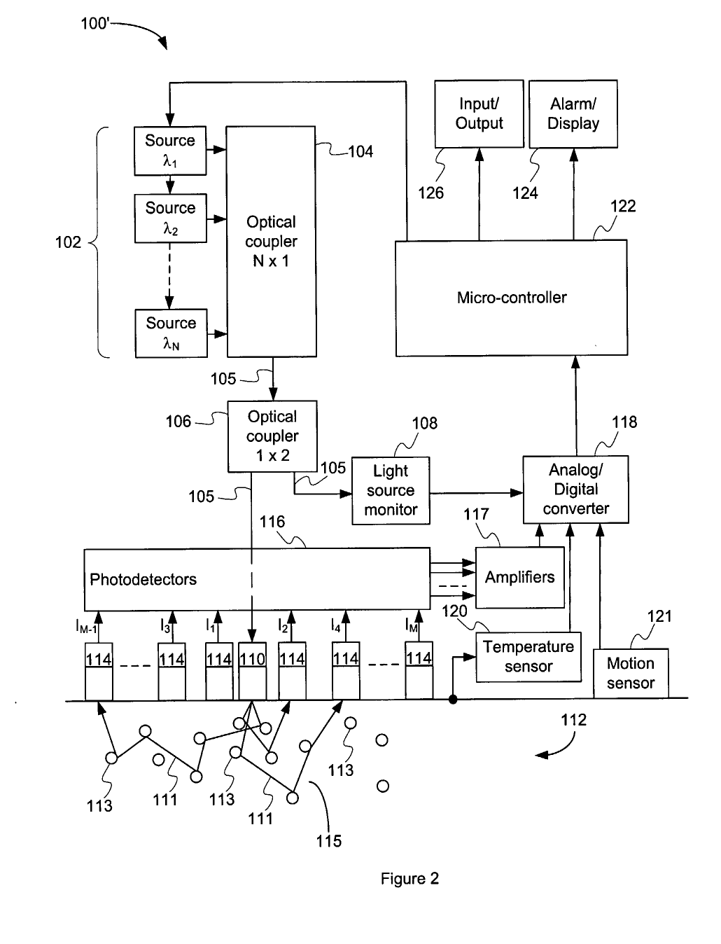

In order to reduce motion artifact caused by, for example, relative movement between the skin and the monitoring device 100 or skin structure deformation, the monitoring device 100 illustrated in FIG. 1 may be modified by adding a motion sensor 121 resulting in the monitoring device 100′ illustrated in FIG. 2. The motion sensor 121, which may be, for example, an accelerometer, a pressure sensor or a combination of both and may be advantageously positioned in contact with the skin. It is to be understood that in the case where the motion sensor 121 is, for example, an accelerometer, it may be positioned at another location within or on the monitoring device 100′.

The ADC 118 then converts the analog signals from the motion sensor 121, into a digital signal which is supplied to the micro-controller 122. The micro-controller 122 algorithm, which controls the operations of the apparatus and performs various computations and estimations according to the applications, then takes into account the information provided by the motion sensor 121.

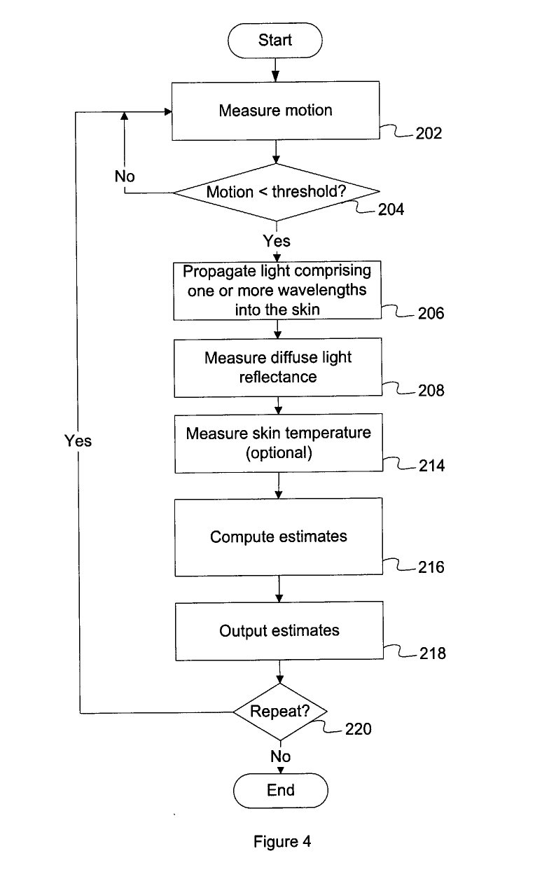

The algorithm previously depicted by the flow chart shown in FIG. 3 may be modified to take into account this new information resulting in the algorithm depicted by the flow chart shown in FIG. 4. The steps composing the algorithm are indicated by blocks 202 to 220.

At block 202 the algorithm starts by measuring the motion of the monitoring device 100′. To that end, many current off the shelf accelerometers and/or pressure sensors may be used for motion sensor 121. Then, at block 204, the algorithm verifies if the measured motion is inferior to a preset threshold value, if so it goes to block 206 and proceeds as per the previous description of the algorithm of FIG. 3, if not, the algorithm goes back to block 202.

Alternatively, in case where the wearer of the monitoring apparatus 100′ is in constant movement above the predetermined motion threshold, a timer or a counter may be added to the algorithm in order to set an alarm to warn the wearer to stand still for a certain period of time in order for the apparatus to proceed with an estimation of the desired physiological parameters.

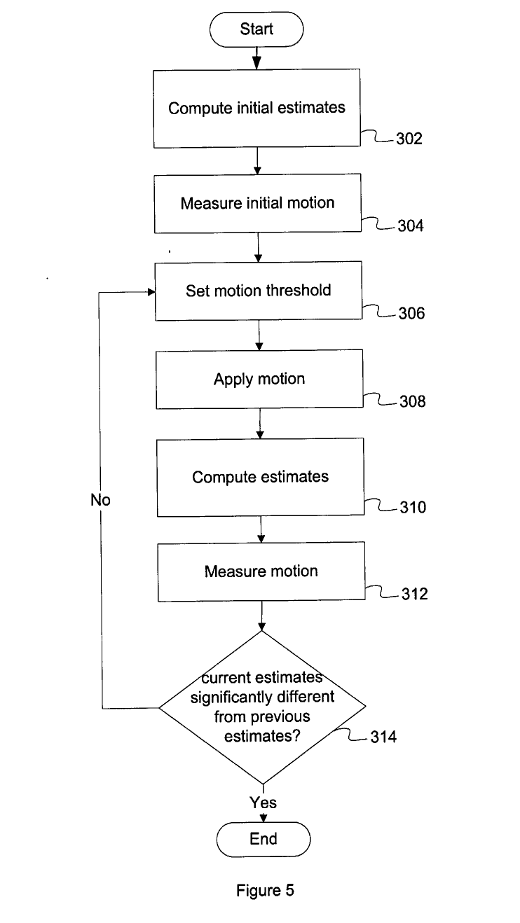

The value of the threshold used at block 204 may be set according to theoretical values or may alternatively be set by the algorithm depicted by the flow chart shown in FIG. 5. The steps composing the algorithm are indicated by blocks 302 to 314.

At block 302 the algorithm starts by computing initial estimates of the desired physiological parameters using, for example, the algorithm depicted by the flow chart shown in FIG. 3. At block 304, the algorithm measures the initial motion value of the monitoring apparatus 100′ and at block 306, sets the motion threshold value to that measured initial value.

Then, at block 308, incremental movement is applied to the monitoring apparatus 100′, following which estimates of the desired physiological parameters are computed at block 310 and a new motion value is measured at block 312.

The algorithm then compares the current parameters estimates to the previous estimates in order to determine if there is a significant difference. If there is a significant difference then the algorithm terminates and returns the value of the motion threshold, if not, the algorithm goes back to block 306 where the motion threshold is set to the current motion value and proceeds to repeat blocks 308 to 314.

The above described motion artifact reduction technique may be used with many other types of measurement apparatuses such as, for example, Oximeters or any other measurement apparatus susceptible to motion.

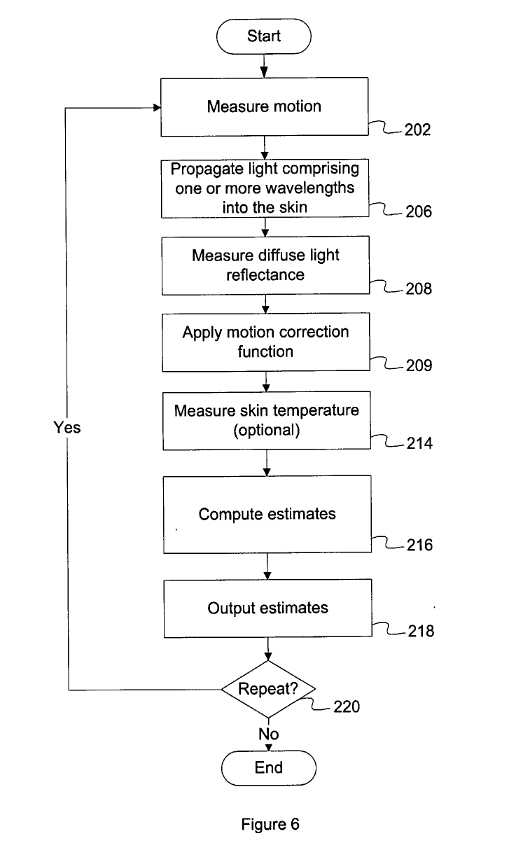

An alternative algorithm to the algorithm depicted by the flow chart shown in FIG. 4 is depicted by the flow chart shown in FIG. 6. The steps composing the algorithm are indicated by blocks 202 to 220.

At block 202 the algorithm starts by measuring the motion of the monitoring device 100′. Then, at block 206, the algorithm propagates light comprising one or more wavelengths into the skin, the wavelengths being selected according to the application of interest such that variations on light reflectance values at the input of the receiver collimating optics 114 may be observed as a function the variation of some estimated parameters.

At block 208, the diffuse light reflectance is measured at two or more distances from the source of the propagated light of block 206. The diffuse light reflectance measurements are advantageously taken simultaneously for all distances, the longer the time interval between each measurement, the less precise the algorithm results may become. The distances, as well as their values, are selected according to the application. The more distances are used, the more precise the diffuse light reflectance model becomes, but also the more computation intensive it becomes and more expensive becomes the associated estimation apparatus 100′.

At block 209 the algorithm applies a motion correction function to the light reflectance measurements made at block 208. The motion correction function is based on the measured motion and is applied in order to compensate for the variation in the measured light reflectance due to the movements of the wearer of the monitoring apparatus 100′.

At block 214, which is optional, the skin temperature is measured.

Then, at block 216, the algorithm computes estimates of the desired physiological parameters, using the corrected reflectance measurements, and skin temperature if measured, and displays those estimates at block 218 using display/alarm 124. The algorithm may further detect clinical conditions using the estimated parameter values, in which case block 218 may also activate an alarm using display/alarm 124. It is to be noted that the parameter estimates and/or detection of clinical conditions may also be provided to another device for further processing using input/output 126. Following which, at block 220, the whole algorithm is repeated if continuous monitoring is desired, otherwise the algorithm ends.

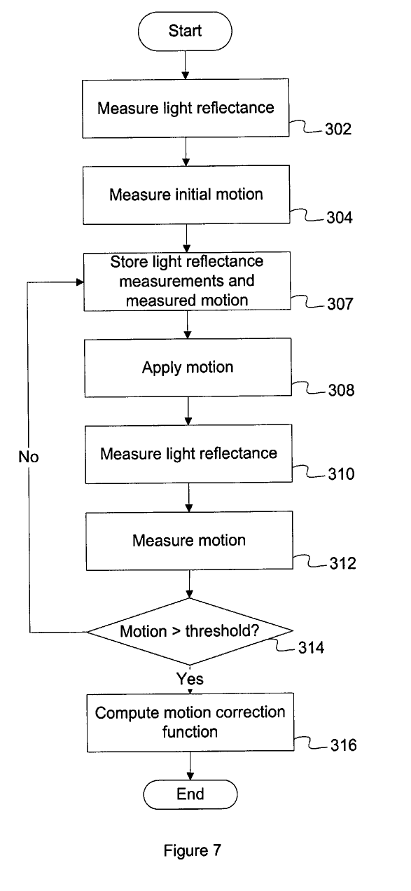

The motion correction function used at block 209 may be set using the algorithm depicted by the flow chart shown in FIG. 7. The steps composing the algorithm are indicated by the blocks 302 to 316.

At block 302 the algorithm starts by measuring the light reflectance by propagating light comprising one or more wavelengths into the skin, the wavelengths being selected according to the application of interest such that variations on light reflectance values at the input of the receiver collimating optics 114 may be observed as a function the variation of some estimated parameters. The diffuse light reflectance is measured at two or more distances from the source of the propagated light. The diffuse light reflectance measurements are advantageously taken simultaneously for all distances, the longer the time interval between each measurement, the less precise the algorithm results may become. The distances, as well as their values, are selected according to the application. At block 304, the algorithm measures the initial motion value of the monitoring apparatus 100′ and at block 307, stores the light reflectance measurements as well as the initial motion value.

Then, at block 308, incremental movement is applied to the monitoring apparatus 100′, following which light reflectance is measured at block 310 and a new motion value is measured at block 312.

The algorithm then compares, at block 314, the measured motion value to a motion threshold. The motion threshold may be set, for example, to a value that is superior to any motion value that may be generated during normal use by a wearer of the monitoring apparatus 100′. If the measured motion value is above the motion threshold, then the algorithm goes to block 316 where a motion correction function is computed using the stored light reflectance measurements and associated measured motion values and then terminates. If the measured motion value is not above the motion threshold, the algorithm goes back to block 307 where the current light reflectance measurements and measured motion value are stored, and proceeds to repeat blocks 308 to 314.

It should be understood that the computation of the motion correction function may be done using any suitable numerical analysis method such as, for example, cubic splines or linear regressions. It should be further understood that if, for example, both an accelerometer and a pressure censor are used, that the threshold may have two components or a single combined component. Furthermore, in the case where the threshold has more than one component, either or all of the measured motion values components may be required to be above or below each corresponding threshold component.

### Spurious Noise Reduction

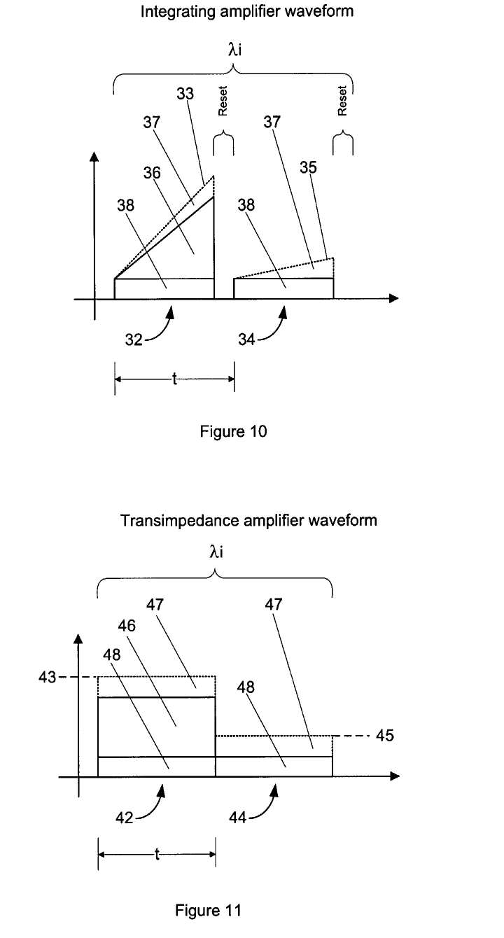

The photodetectors 116 converts the optical signal to an electrical current that will be amplified by amplifiers 117. Two commonly used amplifier technologies are the integrating amplifier and the transimpedance amplifier. FIGS. 10 and 11 show integrating amplifier waveforms and transimpedance amplifier waveforms, respectively, for a given λi.

Referring to FIG. 10, when a signal is emitted by the light sources 102, a first waveform 32 is perceived from the photodetectors 116 using integrating amplifiers. The waveform 32 comprises signal 36, noise 37 and electrical offset 38 components. When no signal is emitted by the light sources 102, a second waveform 34 is perceived from the photodetectors 116, which waveform 34 comprises noise 37 and electrical offset 38 components. The noise 37 component is due, for example, to external lighting conditions which diffuse additional light within the skin and integrated electrical offsets. As for the electrical offset 38 component, it is mainly due to charge transfer during the switching of the integrator and integrator amplifier voltage offsets.

As may be observed, the undesired first waveform 32 components, i.e. the noise 37 and the electrical offset 38 components, may be measured separately from the signal 36 component by taking measurements when the light sources 102 are turned off, i.e. when there is no signal 36 component in the waveform detected by the photodetectors 116.

The signal 36 component may then be recuperated from the first waveforms 32 by subtracting the slope 35 of the second waveform 34 from the slope 33 of the first waveform 32, thus subtracting the noise 37 and the electrical offset 38 components. The slopes 33, 35 may be determined using, for example, least square fitting.

Similarly for photodetectors 116 using transimpedance amplifiers, as shown in FIG. 11, when a signal is emitted by the light sources 102, a first waveform 42 is perceived by the photodetectors 116, which waveform 42 comprises signal 46, noise 47 and electrical offset 48 components. When no signal is emitted by the light sources 102, a second waveform 44 is perceived by the photodetectors 116, which waveform 44 comprises noise 47 and electrical offset 48 components.

As may be observed, the undesired first waveform 42 components, i.e. the noise 47 and the electrical offset 48 components, may be measured separately from the signal 46 component by taking measurements when the light sources 102 are turned off, i.e. when there is no signal 46 component in the waveform detected by the photodetectors 116.

The signal 46 component may then be recuperated from the first waveforms 42 by subtracting the intensity value 45 of the second waveform 44 from the intensity value 43 of the first waveform 42, thus subtracting the noise 47 and the electrical offset 48 components.

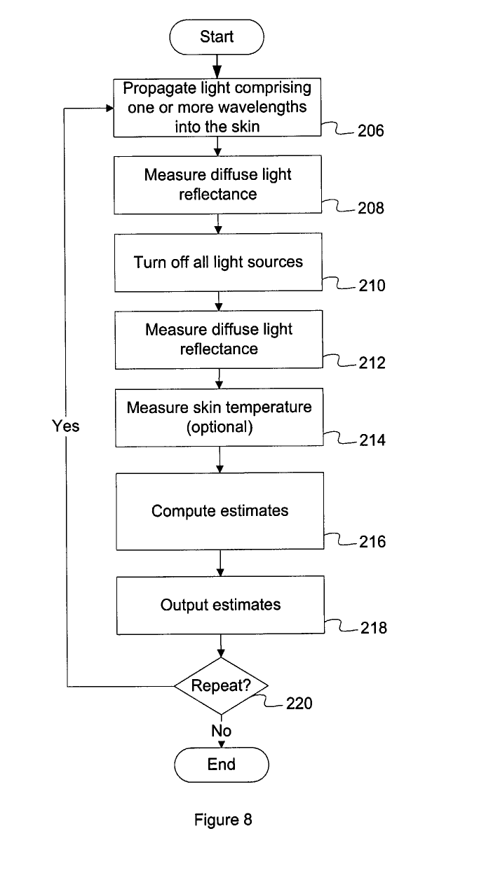

The algorithm previously depicted by the flow chart shown in FIG. 3 may be modified in order to reduce spurious noise present in the reflected beam 111, and possible electrical offsets from the photodetectors 116, resulting in the algorithm depicted by the flow chart shown in FIG. 8. The steps composing the algorithm are indicated by blocks 206 to 220.

At block 206 the algorithm starts by propagating light comprising one or more wavelengths into the skin, the wavelengths being selected according to the application of interest such that variations on light reflectance values at the input of the receiver collimating optics 114 may be observed as a function the variation of some estimated parameters.

At block 208, the diffuse light reflectance is measured at two or more distances from the source of the propagated light of block 206. The diffuse light reflectance measurements are advantageously taken simultaneously for all distances, the longer the time interval between each measurement, the less precise the algorithm results may become. The distances, as well as their values, are selected according to the application. The more distances are used, the more precise the diffuse light reflectance model becomes, but also the more computation intensive is becomes and more expensive becomes the associated estimation apparatus 100.

At block 210, all light sources are turned off so that no light is emitted by the monitoring apparatus 100. The algorithm then measures, at block 212, the diffuse light reflectance as per block 208, providing a measurement of the spurious noise and possible electrical offsets for each wavelength.

At block 214, which is optional, the skin temperature is measured.

Then, at block 216, the algorithm computes adjusted reflectance measurement values by subtracting the measurements taken at block 212 from the measurements taken at block 208, as described above, computes estimates of the desired physiological parameters using the adjusted reflectance measurement values, and skin temperature if measured, and displays those estimates at block 218 using display/alarm 124. The algorithm may further detect clinical conditions using the estimated parameter values, in which case block 118 may also activate an alarm using display/alarm 124. It is to be noted that the parameter estimates and/or detection of clinical conditions may also be provided to another device for further processing using input/output 126. Following which, at block 220, the whole algorithm is repeated if continuous monitoring is desired, otherwise the algorithm ends.

It should be noted that the time during which the diffuse light reflectance is measured, with either the light sources 102 emitting or off, should be kept as small as possible so that the spurious ambient light may not vary substantially between the measurement with the light sources 102 emitting and off.

The above described spurious noise reduction technique may be used with many other types of measurement apparatuses such as optical measurement apparatuses, for example fiber optics Optical Loss Test Sets (OLTS), or Radio Frequency (RF) measurement apparatuses.

### Motion Artifact Reduction and Spurious Noise Reduction

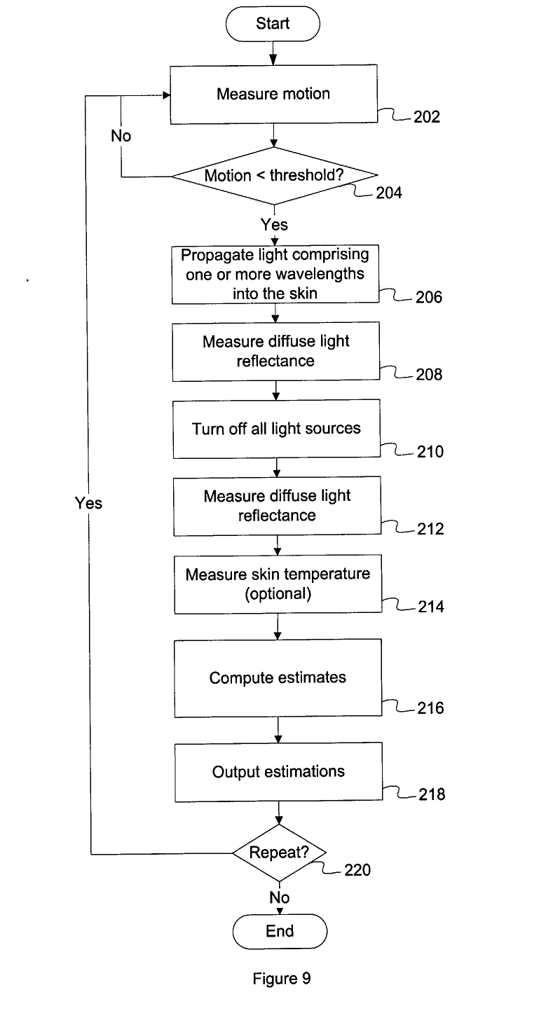

Furthermore, both of the above-described techniques may be combined into a single algorithm depicted by the flow chart shown in FIG. 9. The steps composing the algorithm are indicated by blocks 202 to 220, all of which have been previously described in detail.

Further still, it should be noted that the repetition rate of the samples or the integration period taken for the purpose of the diffuse light reflectance measurements, for a given wavelength, may be chosen so as to be a multiple of the frequency of a parasitic signal, such as, for example, AC line interference. Thus, when the measurements are averaged over a certain number of periods, the effects of the parasitic signal cancel out. For example, an AC line parasitic signal may have a frequency of 60 Hz, so the repetition rate or the integration period of the samples may then be set to 18.75 Hz such that when the measurements are averaged over five periods, this corresponds to 16 periods at 60 Hz. Similarly, averaging the measurements over six periods corresponds to 16 periods at 50 Hz. The two may also be combined such that averaging the measurements over 30 periods corresponds to 96 periods at 60 Hz and 80 periods at 50 Hz, thus canceling out both the 50 Hz and 60 Hz parasitic signals. Of course, the repetition rate or the integration period of the samples may be selected so as to cancel parasitic signals at other frequencies.

Although the present invention has been described by way of non-limitative illustrative embodiments and examples thereof, it should be noted that it will be apparent to persons skilled in the art that modifications may be applied to the present illustrative embodiments without departing from the scope of the present invention.

What is claimed is:

1. A method for reducing motion artifact when computing estimates of values representative of at least one physiological parameter of a subject, comprising the steps of:
measuring a motion value;
comparing the motion value with a motion threshold,
if the compared motion value is lower than the motion threshold then taking at least one physiological measurement;
estimating the values representative of the at least one physiological parameter by applying a mathematical model to the at least one physiological measurement; and
providing the estimate of the values representative of the at least one physiological parameter.

2. A method according to claim 1, wherein the at least one physiological measurement is a reflectance value.

3. A method according to claim 2, wherein the step of taking at least one physiological measurement comprises the further steps of:
generating a probing light beam comprising at least one wavelength;
propagating the probing light beam into the skin of the subject from a propagation point;
measuring reflectance values of the probing light beam from at least two distances from the propagation point.

4. A method according to claim 2, wherein the reflectance values for a given wavelength are measured or integrated at time intervals equal to a multiple of the frequency of a parasitic signal.

5. A method for reducing motion artifact when computing estimates of values representative of at least one physiological parameter of a subject, comprising the steps of:
repeatably measuring a motion value;
comparing each motion value with a motion threshold,
a) if the compared motion value is lower than the motion threshold then
taking at least one physiological measurement;
estimating the values representative of the at least one physiological parameter by applying a mathematical model to the at least one physiological measurement; and
providing the estimates of the values representative of the at least one physiological parameter;
b) if a predetermined number of consecutive compared motion values are higher than the motion threshold then providing a warning to the subject.

6. A method according to claim 5, wherein the at least one physiological measurement is a reflectance value.

7. A method according to claim 6, wherein the step of taking at least one physiological measurement comprises the further steps of:
generating a probing light beam comprising at least one wavelength;
propagating the probing light beam into the skin of the subject from a propagation point;
measuring reflectance values of the probing light beam from at least two distances from the propagation point.

8. A method according to claim 7, wherein the reflectance values for a given wavelength are measured or integrated at time intervals equal to a multiple of the frequency of a parasitic signal.

9. A method for reducing motion artifact when computing estimates of values representative of at least one physiological parameter of a subject, comprising the steps of:
measuring a motion value;
taking at least one physiological measurement;
applying a correction function to the at least one physiological measurement, the correction function being based on the measured motion value;
estimating the values representative of the at least one physiological parameter by applying a mathematical model to the at least one corrected physiological measurement; and
providing the estimates of the values representative of the at least one physiological parameter.

10. A method according to claim 9, wherein the at least one physiological measurement is a reflectance value.

11. A method according to claim 10, wherein the step of taking at least one physiological measurement comprises the further steps of:
generating at a probing light beam comprising at least one wavelength;
propagating the probing light beam into the skin of the subject from a propagation point;
measuring reflectance values of the probing light beam from at least two distances from the propagation point.

12. A method according to claim 11, wherein the reflectance values for a given wavelength are measured or integrated at time intervals equal to a multiple of the frequency of a parasitic signal.

13. A method according to claim 9, wherein the correction function is set by:
incrementally applying motion;
taking at least one physiological measurement;
measuring a motion value associated with the at least one physiological measurement;
comparing each motion value with a motion threshold, if the compared motion value is higher than the motion threshold then compute the correction function using the physiological measurements with associated measured motion values and a numerical analysis method.

14. A method according to claim 13, wherein the numerical analysis method is taken from a group consisting of cubic splines and linear regressions.

15. A method for reducing spurious noise when computing estimates of values representative of at least one physiological parameter of a subject, comprising the steps of:
generating a probing signal comprising at least one wavelength;
propagating the probing signal from a propagation point;
measuring reflectance values of the probing signal for a subset of the at least one wavelength from at least two distances from the propagation point;
shutting off the probing signal for the subset of the at least one wavelength;
measuring a shut-off reflectance value from the at least two distances from the propagation point;
computing adjusted reflectance values by subtracting the shut-off reflectance values from the reflectance values;
estimating the values representative of the at least one physiological parameter by applying a mathematical model to adjusted reflectance values; and
providing the estimates of the values representative of the at least one physiological parameter.

16. A method according to claim 15, wherein the subset of the at least one wavelength comprises all of the wavelengths.

17. A method according to claim 15, wherein the probing signal is a probing light beam.

18. A method according to claim 15, wherein the probing signal is a probing radio frequency.

19. A method according to claim 15, wherein the reflectance values and the shut-off reflectance values for a given wavelength are measured or integrated at time intervals equal to a multiple of the frequency of a parasitic signal.

20. An apparatus for reducing motion artifact when computing estimates of values representative of at least one physiological parameter of a subject, comprising:
emitter for propagating a probing light beam comprising at least one wavelength into the skin of the subject from a propagation point;
at least two receivers for measuring reflectance values of the probing light beam from at least two distances from the propagation point;
a motion sensor;
a display;
a microcontroller operatively connected to the at least two receivers, the motion sensor and the display, wherein the microcontroller comprises an algorithm for:
measuring a motion value using the motion sensor;
comparing the motion value with a motion threshold;
if the compared motion value is lower than the motion threshold then measuring reflectance values using the at least two receivers;
estimating the values representative of the at least one physiological parameter by applying a mathematical model to the reflectance values; and
outputting to the display the values representative of the at least one physiological parameter.

21. An apparatus according to claim 20, wherein the motion sensor is selected from a group consisting of an accelerometer, a pressure sensor and a combination of both.

22. An apparatus according to claim 20, further comprising a temperature sensor for measuring the temperature of the skin of the subject and wherein the mathematical model includes a skin temperature correction factor.

23. An apparatus according to claim 20, wherein the reflectance values are measured or integrated at time intervals equal to a multiple of the frequency of a parasitic signal.

24. An apparatus for reducing motion artifact when computing estimates of values representative of at least one physiological parameter of a subject, comprising:
emitter for propagating a probing light beam comprising at least one wavelength into the skin of the subject from a propagation point;
at least two receivers for measuring reflectance values of the probing light beam from at least two distances from the propagation point;
a motion sensor;
a display;
a microcontroller operatively connected to the at least two receivers, the motion sensor and the display, wherein the microcontroller comprises an algorithm for:
repeatably measuring a motion value using the motion sensor;
comparing each motion value with a motion threshold,
a) if the compared motion value is lower than the motion threshold then
measuring reflectance values using the at least two receivers;
estimating the values representative of the at least one physiological parameter by applying a mathematical model to the reflectance values; and
outputting to the display the values representative of the at least one physiological parameter;
b) if a predetermined number of consecutive compared motion values are higher than the motion threshold then outputting to the display a warning to the subject.

25. An apparatus according to claim 24, wherein the motion sensor is selected from a group consisting of an accelerometer, a pressure sensor and a combination of both.

26. An apparatus according to claim 24, further comprising a temperature sensor for measuring the temperature of the skin of the subject and wherein the mathematical model includes a skin temperature correction factor.

27. An apparatus according to claim 24, wherein the reflectance values are measured or integrated at time intervals equal to a multiple of the frequency of a parasitic signal.

28. An apparatus for reducing spurious noise when computing estimates of values representative of at least one physiological parameter of a subject, comprising:
emitter for propagating a probing signal comprising at least one wavelength into the skin of the subject from a propagation point;
at least two receivers for measuring reflectance values of the probing light beam from at least two distances from the propagation point;
a display;
a microcontroller operatively connected to the at least two receivers and the display, wherein the microcontroller comprises an algorithm for:
measuring reflectance values for a subset of the at least one wavelength using the at least two receivers;
shutting off the probing signal for the subset of the at least one wavelength;
measuring shut-off reflectance values using the at least two receivers;
computing adjusted reflectance values by subtracting the shut-off reflectance values from the reflectance values;
estimating the values representative of the at least one physiological parameter by applying a mathematical model to the adjusted reflectance values; and
outputting to the display the values representative of the at least one physiological parameter.

29. An apparatus according to claim 28, wherein the subset of the at least one wavelength comprises all of the wavelengths.

30. An apparatus according to claim 28, wherein the emitter propagates light and the probing signal is a probing light beam.

31. An apparatus according to claim 28, wherein the emitter propagates radio frequencies and the probing signal is a probing radio frequency.

32. An apparatus according to claim 28, further comprising a temperature sensor for measuring the temperature of the skin of the subject and wherein the mathematical model includes a skin temperature correction factor.

33. An apparatus according to claim 28, wherein the reflectance values are measured or integrated at time intervals equal to a multiple of the frequency of a parasitic signal.
